(19) United States
(12) Patent Application Publication
(10) Pub. No.: US 2026/0073142 A1
(43) Pub. Date: Mar. 12, 2026

(54) SAMPLED LANGUAGE MODELS FOR MEDICAL DECISION MAKING

(71) Applicant: NEC Laboratories America, Inc., Princeton, NJ (US)

(72) Inventor: Christopher A. White, Neshanic Station, NJ (US)

(21) Appl. No.: 19/323,543

(22) Filed: Sep. 9, 2025

Related U.S. Application Data
(60) Provisional application No. 63/692,741, filed on Sep. 10, 2024.

(51) Int. Cl. G06F 40/284; G16H 10/60; G16H 50/20

(52) U.S. Cl. CPC G06F 40/284; G16H 10/60; G16H 50/20

(57) ABSTRACT

Methods and systems include searching for prompt tokens in a document corpus, starting from a random point in the document corpus. A next token is added to an updated prompt from the document corpus after the prompt tokens have been located. The searching and adding are iteratively repeated using the updated prompt until an end condition is reached. An action is performed responsive to the updated prompt.

### RELATED APPLICATION INFORMATION

This application claims priority to U.S. patent application Ser. No. 63/692,741, filed on Sep. 10, 2024, incorporated herein by reference in its entirety.

### BACKGROUND

### Technical Field

The present invention relates to language models and, more particularly, to sampled language models.

### Description of the Related Art

Large language models (LLM) are machine learning models that are implemented using neural network architectures, trained on a large corpus of textual information. LLMs are useful for generating outputs that match the statistical distribution of the training corpus.

### SUMMARY

A method includes searching for prompt tokens in a document corpus, starting from a random point in the document corpus. A next token is added to an updated prompt from the document corpus after the prompt tokens have been located. The searching and adding are iteratively repeated using the updated prompt until an end condition is reached. An action is performed responsive to the updated prompt.

A system includes a hardware processor and a memory that stores a computer program. When executed by the hardware processor, the computer program causes the hardware processor to search for prompt tokens in a document corpus, starting from a random point in the document corpus, to add a next token to an updated prompt from the document corpus after the prompt tokens have been located, to iteratively repeat the searching and adding using the updated prompt until an end condition is reached, and to perform an action responsive to the output tokens.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram illustrating a sampled language model, in accordance with an embodiment of the present invention;

FIG. 2 is a block/flow diagram showing a method for a token search in a sampled language model, in accordance with an embodiment of the present invention;

FIG. 3 is a block/flow diagram showing a method for a token search in a sampled language model, in accordance with an embodiment of the present invention;

FIG. 4 is a block/flow diagram of a method for performing language processing using a sampled language model, in accordance with an embodiment of the present invention;

FIG. 5 is a block diagram of a healthcare facility where a sampled language model is used to perform diagnosis using a document corpus that includes patient medical information, in accordance with an embodiment of the present invention; and

FIG. 6 is a block diagram of a computing device that can implement a sampled language model for patient diagnosis and treatment, in accordance with an embodiment of the present invention.

### DETAILED DESCRIPTION OF PREFERRED EMBODIMENTS

A sampled language model may be implemented by sampling tokens according to the statistics of a corpus of documents. For example, a next token in a conversation may be selected consistent with the preceding context of the conversation (e.g., a prompt) in a manner that does not rely on training a neural network. The sampled language model shifts work from training a neural network to searching the corpus. Instead of modeling the statistical distribution of the corpus, as a large language model (LLM) would do, the sampled language model directly samples tokens from the corpus to obtain results with similar quality.

The sampled language model thus predicts new tokens in a manner that is statistically consistent with a prior context and the corpus of documents. As it is based on a corpus of text documents, and its output depends on the statistical distribution established by those documents, the sampled language model is a form of statistical model that operates in a manner which is distinct from an LLM. A search is performed for the sequence of the prompt, starting from a random point in the corpus of documents. Once the tokens from the prompt, or a sufficiently similar sequence of tokens, have been located, the next token from the corpus is selected as a predicted token. Because the corpus itself is being sampled, the sampled predicted token matches the statistics of the corpus. This search can then be repeated using newly discovered tokens from the corpus, extending the context sequence by adding the previously predicted token to generate a multi-token response. After finding N tokens, the string has a probability consistent with the joint probability of the tokens up to that point P(1, . . . , N).

As the number of tokens increases, it is less likely that tokens in the correct order will be found within the existing corpus. To address this, the sampled language model uses a large corpus and uses a fuzzy search where the closest match to the previous tokens is acceptable, rather than requiring an exact match to the context sequence.

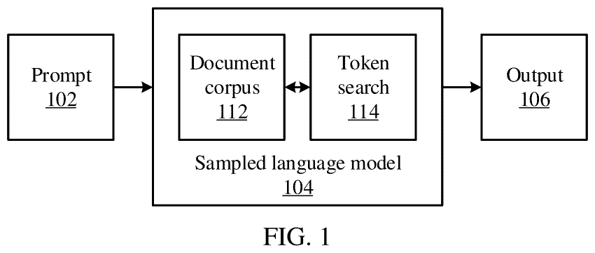

Referring now to FIG. 1, token generation with a sampled language model is shown. A prompt 102 is provided to the sampled language model 104, which generates one or more predicted tokens as its output 106. The sampled language model 104 performs a token search on a document corpus 112, looking for tokens from the prompt 102, and then selects the predicted tokens from the document corpus 112 to generate the output. The term “tokens,” as used herein, may refer to words, individual characters, or any other appropriate subdivision of language within the document corpus 112.

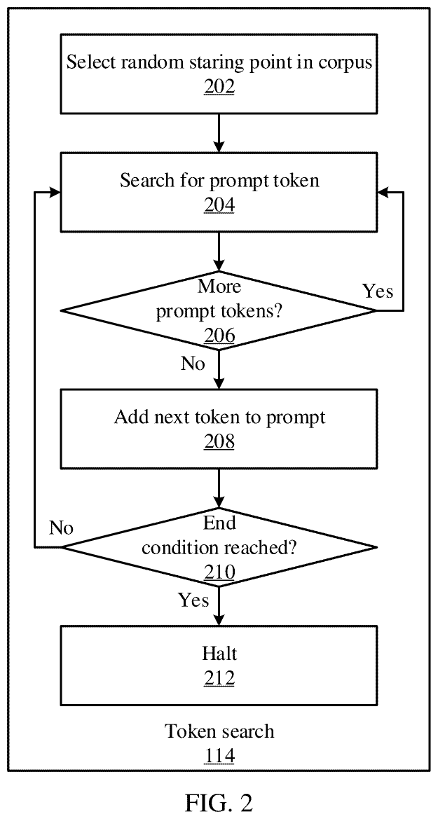

Referring now to FIG. 2, a method for performing token search 114 is shown. Block 202 begins by selecting a random starting point in the document corpus 112. Block 204 then begins to search forward through the document corpus 112, token by token, until arriving at a token that matches the first token of the prompt 102. This begins a loop, where block 206 determines whether there are additional tokens left in the prompt 102. If so, block 204 continues to search through the corpus until the next token from the prompt 102 is found, skipping any tokens which do not match. If the end of the document corpus 112 is reached, the search may begin again from an initial point in the document corpus 112.

Once there are no further tokens in the prompt 102, block 208 outputs the next token from the document corpus 112 and adds it to the prompt. Block 210 determines whether an end condition has been satisfied. If not, block 208 adds the next token from the document corpus 112 to the prompt and the search begins again. Once the end condition has been reached, block 212 halts the token search 114, and the output 106 is complete, including the new tokens. In this manner, rather than building an approximate representation of the statistics of a corpus of documents, the sampled language model 104 samples a sequence of tokens directly from the document corpus 112. This represents just one exemplary method for performing the token search 114.

The end condition of block 210 may depend on the nature of the corpus. LLMs may be trained with segments that have an “end of response” token, and such a token can similarly be used as the end condition here. In some embodiments, the end condition may specify a number of N last tokens of the search are matched, which would address situations where the whole sequence cannot be found. In such embodiments, the number of possible options for the search may be considered. As the number increases, the likelihood of finding irrelevant material increases.

In some embodiments, the search 204 may be performed by a machine learning system. In such embodiments, the machine learning system may be trained to improve the quality of the search's output. For example, take prompts & responses from an LLM may be used to train the search model to replicate the quality of the original LLM.

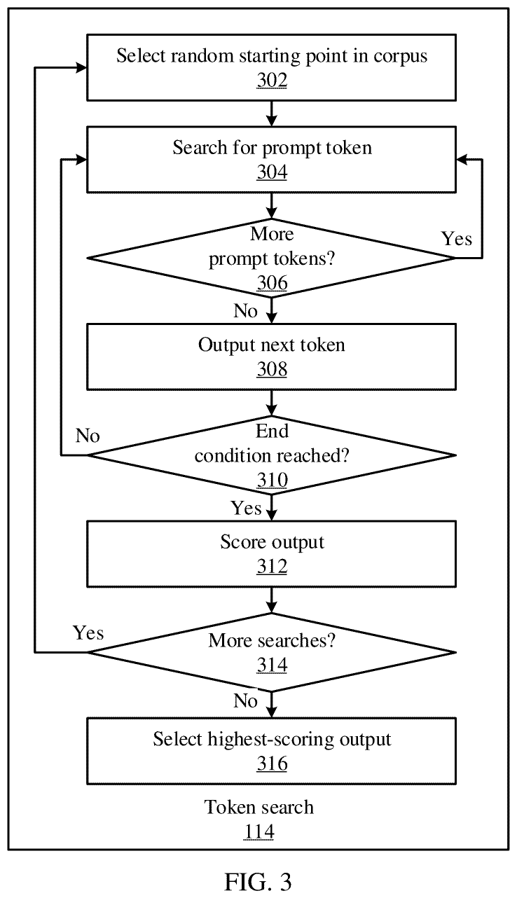

Referring now to FIG. 3, the token search 114 may perform multiple distinct searches, starting from different random points in the document corpus 112. The search process is similar to that of FIG. 2, but the search of block 304 may search in both directions (forward and backward in the document corpus 112) from the random starting point selected by block 302. As above, block 306 determines whether there are more prompt tokens, and returns processing to block 304. Some embodiments may search a maximum of N tokens away from the previous starting point for a prompt token and, if the next prompt token is not found within those N tokens, then the search may continue from that previous starting point with a next token from the prompt 102. Once the sequence of the prompt 102 has been exhausted, block 308 adds the next token from the document corpus 112 to the prompt and the search begins again, iterating until an end condition is reached at block 310.

Searches may be compared to one another based on a score that is related to distances traversed to find the prompt tokens. Block 312 thus scores the output of each search. If block 314 determines that another search is to be performed, processing returns to block 302 and a new random starting point is selected. In some embodiments, the multiple searches may be performed and scored in parallel. Block 316 selects the highest-scoring output to use as output 106.

The scoring function may be based on the difficulty of finding a match. For example, an exemplary scoring function may look to the largest number of words that match. So an exact match might have the best score. Another exemplary scoring function may be to search one word at a time and, for each word, going forward or backward as many tokens as needed to find the next token. Such a scoring function could take the distances needed to find next tokens, summed across the entire string. In such a function an exact match in the corpus to the prompt sequence would provide the lowest search distance and a best score, while a match that needed to traverse many tokens would have a worst score.

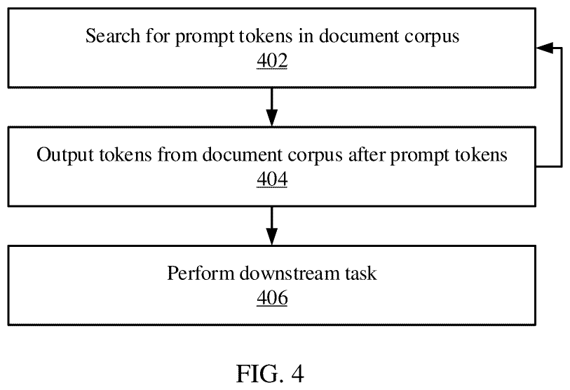

Referring now to FIG. 4, a method of using a sampled language model is shown. Block 402 searches for the prompt's tokens within the document corpus. As described above, this search may start from a random initial point and may proceed in one or both directions to find each token from the prompt in turn. Once the tokens from the prompt 102 are exhausted, block 404 finds a next token from the document corpus to add to the prompt, and the search 402 repeats using the updated prompt until an end condition is reached. Block 406 then performs a downstream task using the output including the original prompt and the discovered tokens.

The downstream task may be any appropriate language generating task, for example performing question answering based on a corpus of domain-specific knowledge. The document corpus 112 may include text documents that establish the norms for general purpose language generation. Domain-specific documents may be added to the document corpus 112, so that prompts relating to that domain may be answered accurately. In some embodiments, the document corpus 112 may further include private or proprietary documents that are not publicly available. The document corpus 112 may thereby be modified to adapt the question answering system to any appropriate domain without the need for model retraining or fine-tuning. As long as the document corpus 112 reflects the statistical distribution of language appropriate to the target domain, the token search 114 will generate text appropriate to that domain.

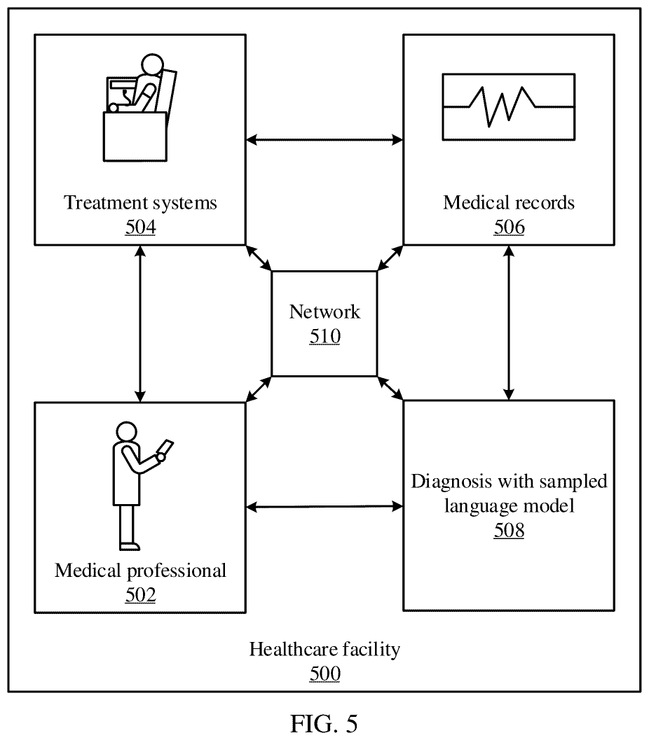

Referring now to FIG. 5, a diagram of RAG-based solutions to health issues is shown in the context of a healthcare facility 500. Diagnosis with a sampled language model 508 may be used to process information relating to a patient's health condition, for example based on the patient's medical records 506 and general information relating to medical conditions. The sampled language model 508 may be based on a corpus that includes domain-specific information, for example relating to a medical specialty or a patient's own medical records.

The healthcare facility may include one or more medical professionals 502 who review information extracted from a patient's medical records 506 to determine their healthcare and treatment needs. These medical records 506 may include self-reported information from the patient, test results, and notes by healthcare personnel made to the patient's file. Treatment systems 504 may furthermore monitor patient status to generate medical records 506 and may be designed to automatically administer and adjust treatments as needed.

Based on information drawn from the diagnosis with sampled language model 508, the medical professionals 502 may then make medical decisions about patient healthcare suited to the patient's needs. For example, the medical professionals 502 may make treatment decisions based on a diagnosis generated by the diagnosis with sampled language model 508 and may prescribe particular medications, surgeries, and/or therapies that are appropriate to the diagnosis disease.

The different elements of the healthcare facility 500 may communicate with one another via a network 510, for example using any appropriate wired or wireless communications protocol and medium. Thus diagnosis with sampled language model 508 receives data from treatment systems 504, medical professionals 502, and from medical records 506, and generates an output that specifies a diagnosis and/or treatment for the patient. The diagnosis with sampled language model 508 may further coordinate with treatment systems 504 in some cases to automatically administer or alter a treatment. For example, if the output indicates a particular treatment, the system may automatically trigger implementation of the treatment, such as by initiating or halting the administration of a medication.

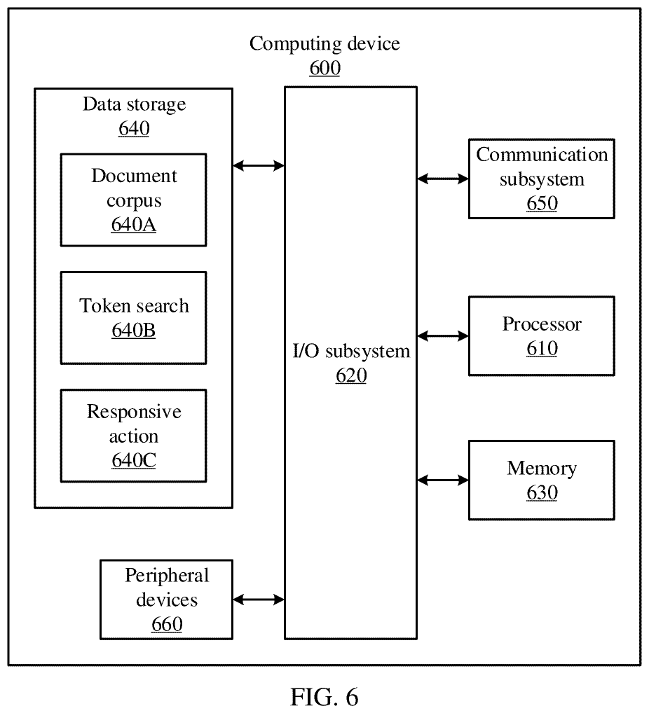

Referring now to FIG. 6, an exemplary computing device 600 is shown, in accordance with an embodiment of the present invention. The computing device 600 is configured to perform visual question answering.

The computing device 600 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 600 may be embodied as one or more compute sleds, memory sleds, or other racks, sleds, computing chassis, or other components of a physically disaggregated computing device.

As shown in FIG. 6, the computing device 600 illustratively includes the processor 610, an input/output subsystem 620, a memory 630, a data storage device 640, and a communication subsystem 650, and/or other components and devices commonly found in a server or similar computing device. The computing device 600 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 630, or portions thereof, may be incorporated in the processor 610 in some embodiments.

The processor 610 may be embodied as any type of processor capable of performing the functions described herein. The processor 610 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 630 may be embodied as any type of volatile or non-volatile memory or data storage capable of performing the functions described herein. In operation, the memory 630 may store various data and software used during operation of the computing device 600, such as operating systems, applications, programs, libraries, and drivers. The memory 630 is communicatively coupled to the processor 610 via the I/O subsystem 620, which may be embodied as circuitry and/or components to facilitate input/output operations with the processor 610, the memory 630, and other components of the computing device 600. For example, the I/O subsystem 620 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 620 may form a portion of a system-on-a-chip (SOC) and be incorporated, along with the processor 610, the memory 630, and other components of the computing device 600, on a single integrated circuit chip.

The data storage device 640 may be embodied as any type of device or devices configured for short-term or long-term storage of data such as, for example, memory devices and circuits, memory cards, hard disk drives, solid state drives, or other data storage devices. The data storage device 640 can store program code 640A for a document corpus, 640B for searching tokens, and/or 640C for performing responsive actions. Any or all of these program code blocks may be included in a given computing system. The communication subsystem 650 of the computing device 600 may be embodied as any network interface controller or other communication circuit, device, or collection thereof, capable of enabling communications between the computing device 600 and other remote devices over a network. The communication subsystem 650 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 600 may also include one or more peripheral devices 660. The peripheral devices 660 may include any number of additional input/output devices, interface devices, and/or other peripheral devices. For example, in some embodiments, the peripheral devices 660 may include a display, touch screen, graphics circuitry, keyboard, mouse, speaker system, microphone, network interface, and/or other input/output devices, interface devices, and/or peripheral devices.

Of course, the computing device 600 may also include other elements (not shown), as readily contemplated by one of skill in the art, as well as omit certain elements. For example, various other sensors, input devices, and/or output devices can be included in computing device 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 600 are readily contemplated by one of ordinary skill in the art given the teachings of the present invention provided herein.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor-or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
searching for prompt tokens in a document corpus, starting from a random point in the document corpus;
adding a next token to an updated prompt from the document corpus after the prompt tokens have been located;
iteratively repeating the searching and adding using the updated prompt until an end condition is reached; and
performing an action responsive to the updated prompt.

2. The method of claim 1, wherein searching for prompt tokens includes performing multiple searches from multiple different random starting points in the document corpus.

3. The method of claim 2, wherein searching for prompt tokens includes scoring each of the multiple searches according to a cumulative distance from respective random starting point to the prompt tokens.

4. The method of claim 3, wherein the action is performed responsive to the updated prompt from a search of the multiple searches having a highest score.

5. The method of claim 2, wherein each of the multiple searches is limited to a predetermined range of tokens around the respective random starting point.

6. The method of claim 1, wherein searching for prompt tokens includes searching for each of a sequence of prompt tokens in order, skipping tokens of the document corpus that do not match.

7. The method of claim 1, wherein the document corpus includes a domain-specific documents relating to a medical specialty.

8. The method of claim 1, wherein searching for prompt tokens is performed using a machine learning system.

9. The method of claim 1, wherein the document corpus includes medical information relating to a patient's condition and wherein the action includes a treatment action to treat the patient's condition.

10. The method of claim 7, wherein the output tokens include a diagnosis to assist in medical decision making.

11. A system, comprising:
a hardware processor; and
a memory that stores a computer program which, when executed by the hardware processor, causes the hardware processor to:
search for prompt tokens in a document corpus, starting from a random point in the document corpus;
add a next token to an updated prompt from the document corpus after the prompt tokens have been located; and
iteratively repeat the searching and adding using the updated prompt until an end condition is reached; and
perform an action responsive to the updated prompt.

12. The system of claim 11, wherein the search for prompt tokens includes performing multiple searches from multiple different random starting points in the document corpus.

13. The system of claim 12, wherein the search for prompt tokens includes scoring each of the multiple searches according to a cumulative distance from respective random starting point to the prompt tokens.

14. The system of claim 13, wherein the action is performed responsive to the updated prompt from a search of the multiple searches having a highest score.

15. The system of claim 12, wherein each of the multiple searches is limited to a predetermined range of tokens around the respective random starting point.

16. The system of claim 11, wherein the search for prompt tokens includes searching for each of a sequence of prompt tokens in order, skipping tokens of the document corpus that do not match.

17. The system of claim 11, wherein the document corpus includes a domain-specific documents relating to a medical specialty.

18. The system of claim 11, wherein the search for prompt tokens is performed using a machine learning system.

19. The system of claim 11, wherein the document corpus includes medical information relating to a patient's condition and wherein the action includes a treatment action to treat the patient's condition.

20. The system of claim 17, wherein the output tokens include a diagnosis to assist in medical decision making.
